(19) United States
(12) Patent Application Publication
(10) Pub. No.: US 2010/0145189 A1
(43) Pub. Date: Jun. 10, 2010

(54) Intraoral scanner

(76) Inventor: Josef Hintersehr, 64347 Griesheim (DE)

Correspondence Address: DeMont & Breyer, LLC, 100 Commons Way, Ste. 250, Holmdel, NJ, 07733, US

(21) Appl. No.: 12/582,192

(22) Filed: Oct. 20, 2009

(30) Foreign Application Priority Data
Dec. 9, 2008 (DE) 10 2008 060 861.0

(51) Int. Cl. A61B 5/05

(52) U.S. Cl. 600/425

(57) ABSTRACT

The invention relates to an intraoral scanner for collecting three-dimensional measured or scanned data of the jaw or teeth, which accommodates a scanning unit in a front region leading into the oral region and which at this front region also accommodates an air delivery device through which pressurized air may be locally supplied in the oral region.

### FIELD OF INVENTION

The invention relates to an intraoral scanner.

### BACKGROUND OF THE INVENTION

Intraoral scanners are known, for example in the form of a pinlike scanner which may be inserted into the oral region.

A scanning unit, for example a laser scanning unit, is typically accommodated in a front region of the scanner, which collects three-dimensional measured or scanned data of the jaw or teeth, etc. in the oral region, the scanner being connected to a data processing unit, located outside the oral region, to which the collected measured or scanned data are transmitted for further, typically computer-aided design (CAD)-based, data processing.

These types of scanners are used in particular for applications in which such three-dimensional jaw and/or teeth data are processed with computer assistance to create a CAD model of a desired dental component such as an implant and restoration of a crown, or bridge and bridge framework, and/or for generating CAD model layers for processing layer data which may be used in a milling, laser sintering, or laser melting process.

For such category-defining intraoral scanners it has proven to be difficult to collect valid three-dimensional measured or scanned data of the jaw and/or teeth in transition regions with respect to the buccal or dental gingiva, for example because this region conceals the view of a subgingival preparation.

### SUMMARY OF THE INVENTION

The object of the invention, therefore, is to refine such a category-defining intraoral scanner for collecting three-dimensional measured or scanned data within the oral region so as to be more effective, in particular to improve the quality of collection of three-dimensional measured or scanned data of the jaw and/or teeth in transition regions with respect to the buccal or dental gingiva by displacing the gingiva and thus exposing the subgingival preparation. A further advantage is the removal of any saliva or discharged blood that may be present, or to minimize interference therefrom.

Accordingly, an intraoral scanner for collecting three-dimensional measured or scanned data of the jaw or teeth is provided which accommodates a scanning unit in a front region leading into the oral region and which at this front region also accommodates an air delivery device through which pressurized air may be locally supplied in the oral region. In this manner, in particular in transition regions between hard, inflexible zones and soft zones, a pocket or type of groove may be formed as a result of the supplied pressurized air so that, also in these transition regions, valid collection of three-dimensional measured or scanned data is enabled in hard, inflexible zones such as the jaw and/or teeth in particular, since these regions are made clearly visible to the camera.

### BRIEF DESCRIPTION OF THE DRAWINGS

The invention is described in greater detail below by means of one exemplary embodiment, with reference to the drawings, which show the following:

FIG. 1 shows a greatly simplified illustration of one embodiment of the invention during use, and

FIG. 2 shows a greatly simplified, enlarged view of a partial region of FIG. 1.

### DETAILED DESCRIPTION

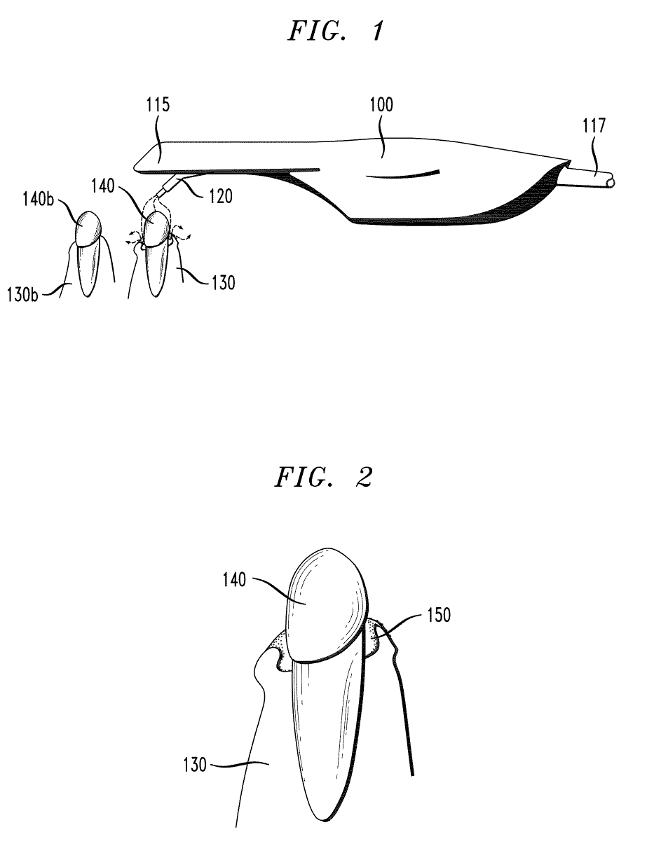

FIG. 1 shows an intraoral scanner 100, having a front region 115 which is inserted into an oral region (not further illustrated) in order to collect three-dimensional measured or scanned data at that location—in the case illustrated, of teeth 140, 140b.

To this end, a scanning unit, for example a laser scanning unit (not further illustrated), is accommodated in the front region 115 of the scanner 100 to be inserted into the oral region. This scanning unit may be used to collect three-dimensional measured or scanned data of the jaw or teeth, etc., in a manner known as such to one skilled in the art, by appropriate manipulation of the scanner 100.

As shown in FIG. 1, also accommodated at the front region 115 is an air delivery device 120 through which pressurized air may be locally supplied in the oral region. The air is supplied from outside the scanner 100 via a connecting line 117 in which at least one type of tube is accordingly integrated.

The air delivery device 120 causes an air stream, which advantageously may be controlled in such a way that the air stream may diffuse around the entire periphery of the region of a given hard, inflexible zone and thus also in transition regions between hard, inflexible zones and soft zones, to form a pocket or type of groove as a result of the supplied pressurized air so that, also in such transition regions, valid collection of three-dimensional measured or scanned data is enabled in hard, inflexible zones such as the jaw and/or teeth in particular.

It is shown in FIG. 1 that the air delivery device 120 causes at least one air stream to diffuse around the entire periphery of the region of a portion of a tooth 140 located outside the gingiva 130. FIG. 1 indicates three nozzles from which three individual air streams flow, and which as a whole essentially diffuse around the entire periphery of this region.

As shown in particular in FIG. 2, a type of pocket or groove 150 is thus formed between the gingiva 130 and the tooth 140, so that also the transition region, which is normally covered by the tooth 140 via the gingiva 130, may be recorded by the scanning unit of the scanner 100. In other words, due to the supplied air pressure the gingiva 130 are pushed away slightly from the tooth 140, thus forming the pocket or groove 150 and exposing the tooth 140 at this location.

Such a pocket is not present, for example, in the transition region between the tooth 140b and the gingiva 130b according to FIG. 1.

In particular to control the air pressure in a targeted manner, for example to simultaneously form a pocket all the way around a tooth or to form larger and smaller pockets, it is practical for the air delivery device to be connected to a control device by means of which the quantity and/or pressure of the air supply may be adjusted.

For the targeted control of a given locally selectable oral region, it is also practical for the air delivery device to have at least one nozzle which is adjustable in position for the targeted delivery of air.

Thus, for example, a zone may be subjected to air flow in a targeted manner, i.e., locally defined and concentrated. This is the case, for example, for tooth 140 according to FIG. 1, whereas tooth 140b is largely spared from the air stream.

Such an adjustment of the at least one nozzle may be performed manually, for example before the intraoral scanning, depending on the particular design of the nozzle, or by use of a control device which is connected to the air delivery device. In the latter case, for example, the nozzles are movably attached to the scanner via actuators, and additional lines for controlling the actuators are integrated into the connecting line 117.

A further advantage of the air delivery is that any saliva or discharged blood present is removed, or its interference is minimized.

The scanner 100 is also connected to a data processing unit (not further illustrated) located outside the oral region, in the case shown, for example, via lines additionally integrated into the connecting line 117 to which the collected measured or scanned data are transmitted for further computer-aided design (CAD)-based data processing. However, a wireless connection may be provided instead of a wired connection between the scanner 100 and the data processing unit. In principle, it is also not necessary for such a connection to be permanent; rather, a memory, for example, may be provided in the scanner from which the stored data may be read out after the intraoral scanning.

1. Intraoral scanner for collecting three-dimensional measured or scanned data of a jaw or teeth in an oral region, the intraoral scanner comprising:
a scanning unit in a front region of the intraoral scanner leading into the oral region; and
at the front region, an air delivery device through which pressurized air may be locally supplied in the oral region.

2. Intraoral scanner according to claim 1, wherein the intraoral scanner is connected by a connection to a data processing unit, located outside the oral region, to which the collected measured or scanned data are transmitted for further computer-aided design (CAD)-based data processing.

3. Intraoral scanner according to claim 2, wherein the connection is wired.

4. Intraoral scanner according to claim 1, wherein the air delivery device is connected to a control device by means of which quantity and/or pressure of the air supply may be adjusted.

5. Intraoral scanner according to claim 1, wherein the air delivery device has at least one nozzle which is adjustable in position for targeted delivery of air.

6. Intraoral scanner according to claim 5, wherein the at least one nozzle may be adjusted manually or by use of a control device which is connected to the air delivery device.

7. Intraoral scanner according to claim 2, wherein the connection is wireless.
